(19) United States
(12) Patent Application Publication
(10) Pub. No.: US 2021/0130671 A1
(43) Pub. Date: May 6, 2021

(54) COLD STORAGE MATERIAL AND COLD STORAGE PACK

(71) Applicant: SHARP KABUSHIKI KAISHA, Sakai City, Osaka (JP)

(72) Inventors: DAISUKE SHINOZAKI, Sakai City, Osaka (JP); KYOHEI SEZUKURI, Sakai City, Osaka (JP); SATORU MOTONAMI, Sakai City, Osaka (JP); YUKA UTSUMI, Sakai City, Osaka (JP); MASAKAZU KAMURA, Sakai City, Osaka (JP)

(21) Appl. No.: 16/625,645

(22) PCT Filed: Jun. 22, 2018
(86) PCT No.: PCT/JP2018/023856

§ 371 Date: Dec. 20, 2019

(30) Foreign Application Priority Data
Jun. 22, 2017 (JP) 2017-122161

(51) Int. Cl. C09K 5/06; A61F 7/10

(52) U.S. Cl. CPC C09K 5/066; A61F 2007/108; A61F 7/10

(57) ABSTRACT

A cold storage material and a cold storage pack are provided where a single cooling medium (cold insulation pack) containing a cold storage material having a plurality of melting points can keep an object at a low temperature that is suited to a situation. The cold storage material changes phase at a prescribed temperature and contains: water; a base compound containing a quaternary ammonium salt that forms a semi-clathrate hydrate; and a supercooling inhibitor that suppresses supercooling, wherein the cold storage material has one or more melting points depending on a temperature range in which the cold storage material has been frozen. These features allow the user to set the melting point of the cold storage material as he/she wants, by properly changing the freezing temperature for intended usage

### TECHNICAL FIELD

The present invention, in some aspects thereof, relates to cold storage materials that change at a prescribed temperature and also to cold storage packs prepared using such a cold storage material.

The present application claims priority to Japanese Patent Application, Tokugan, No. 2017-122161 filed in Japan on Jun. 22, 2017, the contents of which are incorporated herein by reference.

### BACKGROUND ART

Clathrate hydrates, semi-clathrate hydrates in particular, crystallize when an aqueous solution of their base compound is cooled below a temperature at which a hydrate is formed (see FIG. 16). Crystals will store thermal energy that may later be utilized as latent heat. The clathrate hydrate has therefore been used as a latent thermal storage material or as a component of such a material.

Substances worth a mention here are hydrates of quaternary ammonium salts, which are typical examples of semi-clathrate hydrates encaging a non-gaseous species as a guest compound. These hydrates are formed under normal pressure, give out a large amount of thermal energy (amount of stored heat) upon crystallization, and are, unlike paraffin, non-flammable. Therefore, hydrates of quaternary ammonium salts are easy to handle and for this reason attracting attention as a replacement of ice thermal storage tanks in air conditioning systems for buildings.

Among these materials, the semi-clathrate hydrate encaging tetra-n-butylammonium bromide or tri-n-butyl-n-pentylammonium bromide as a guest has latent heat the thermal energy of which is available for use at temperatures higher than the temperature at which the thermal energy of the latent heat of ice becomes available for use. Therefore, the semi-clathrate hydrate has been increasingly used in thermal storage tanks and heat transport media that are more efficient than ice thermal storage tanks.

Cooling therapies have been known such as icing and cryotherapy. Cooling therapy cools the entire human body or hot parts of the body, for example, by blowing cold air at the human body or by placing a cooling medium in contact with the skin of the human body.

Physical exercises and activities in intense heat may induce rises in body temperature, which in turn can lead to decreased performance and increased heatstroke risk. There is a report about cooling of the body before and during a physical exercise or activity in such intense heat. According to the report, for example, cooling before a physical exercise or activity reduces heat strain during an aerobic exercise and improves performance in the exercise in intense heat (Precooling methods and their effects on athletic performance: A systematic review and practical applications, by Ross M, Abbiss C, Laursen P, Martin D, and Burke L, Sports Med. 2013; 43: 207-225). In other words, as cooling before a physical exercise or activity in intense heat, the body is preferably cooled using a cooling medium that has a temperature range in which the body can be gently cooled.

Patent Literature 1 discloses a cooling medium that is expected to provide increased comfort and fittedness and deliver sufficient cooling performance when worn around the human head. This cooling medium includes a plurality of horizontally coupled coupling media having a thickness of 15 to 35 mm and a non-freezing medium having a thickness of 5 to 15 mm. These media are stacked and contained in an exterior bag.

### CITATION LIST

### Patent Literature

Patent Literature 1: Japanese Unexamined Patent Application Publication, Tokukaihei, No. 7-95998

### SUMMARY OF INVENTION

### Technical Problem

If a plurality of temperature ranges is to be used in different manners in cooling the human body or another object to be kept cold, a plurality of cooling media is needed to provide the plurality of temperature ranges. Cited Document 1 discloses a cooling medium that is intended to be used on the human body, but falls short of describing a single cooling medium being used in a plurality of different temperature ranges depending on a situation.

The present invention, in one aspect thereof, has been made in view of these issues and has an object to provide a cold storage material and a cold storage pack, where a single cooling medium (cold insulation pack) containing a cold storage material having a plurality of melting points can keep an object at a low temperature that is suited to a situation.

### Solution to Problem

To achieve the object described above, the present invention takes the following measures. The present invention, in one aspect thereof, is directed to a cold storage material changing phase at a prescribed temperature, the cold storage material containing: water; a base compound containing a quaternary ammonium salt that forms a semi-clathrate hydrate; and a supercooling inhibitor that suppresses supercooling, wherein the cold storage material has one or more melting points depending on a temperature range in which the cold storage material has been frozen.

### Advantageous Effects of Invention

The present invention, in some aspects thereof, provides a cold storage material that has a plurality of different melting points, thereby enabling a single cold insulation pack (cooling medium) to keep an object at low temperatures that are suited to various situations.

### BRIEF DESCRIPTION OF DRAWINGS

FIG. 1 is a schematic illustration of procedures of comparing freezing temperatures.

FIG. 2 is a graph representing changes in temperature of a cold storage material obtained in Example 1.

FIG. 3 is a graph representing results of a DSC experiment on the cold storage material obtained in Example 1.

FIG. 4 is a schematic illustration of the arrangement of a high-melting-point component and a low-melting-point component resulting from different freezing temperatures.

FIG. 5 is a graph representing changes in temperature of the cold storage material obtained in Example 1.

FIG. 6 is a graph representing changes in temperature of a cold storage material obtained in Example 6.

FIG. 7 is a graph representing results of a DSC experiment on cold storage materials obtained in Examples 1 to 5.

FIG. 8 is a graph representing results of a DSC experiment on cold storage materials obtained in Examples 6 to 10.

FIG. 9A is a schematic perspective view of a cold storage pack in accordance with a second embodiment.

FIG. 9B is a cross-sectional view taken along line 9b-9b in FIG. 9A.

FIG. 9C is a schematic perspective view of a cold storage pack including articulation mechanisms in accordance with the second embodiment.

FIG. 10 is a schematic perspective view of Variation Example 1 of the cold storage pack in accordance with the second embodiment.

FIG. 11 is a schematic perspective view of Variation Example 2 of the cold storage pack in accordance with the second embodiment.

FIG. 12 is a schematic perspective view of Variation Example 3 of the cold storage pack in accordance with the second embodiment.

FIG. 13 is a schematic perspective view of Variation Example 3 of the cold storage pack in accordance with the second embodiment.

FIG. 14 is a schematic perspective view of a cold storage pack in accordance with a third embodiment.

FIG. 15 is a diagram representing examples of color changes of a thermochromic substance with the temperature of a cold storage material.

FIG. 16 is an illustration of crystallization of a semi-clathrate hydrate.

FIG. 17 is a diagram representing the melting behavior of a cold storage material for different freezing temperatures.

### DESCRIPTION OF EMBODIMENTS

The inventors of the present invention have focused on the fact that a single cold insulation pack (cooling medium) is conventionally incapable of maintaining an object in a plurality of different, low temperature ranges in accordance with various situations and found that a cold storage material is of such a nature that it comes to have one or more melting points if adjusted properly in composition and frozen in a proper temperature range. The inventors have also found that such a cold insulation pack (cooling medium), even if used singly, can maintain an object in a plurality of different, low temperature ranges in accordance with various situations, which has led to the completion of the present invention.

Specifically, the present invention, in some aspects thereof, is directed to a cold storage material changing phase at a prescribed temperature, the cold storage material including: water; a base compound containing a quaternary ammonium salt that forms a semi-clathrate hydrate; and a supercooling inhibitor that suppresses supercooling, wherein the cold storage material has one or more melting points depending on a temperature range in which the cold storage material has been frozen.

Accordingly, the inventors have made it possible to maintain an object at a low temperature that is suited to a situation, by using a single cold insulation pack (cooling medium) containing a cold storage material that changes phase at a plurality of different melting points.

The following will give definitions of some terms used in the present application. These terms should be construed in conformity with the definitions unless otherwise mentioned.

(1) The terms, “clathrate hydrates” and “semi-clathrate hydrates,” are used interchangeably. The present invention, in one aspect thereof, is directed to hydrates encaging a non-gaseous species as a guest (guest compound).

(2) The terms, “thermal storage material” and “cold storage material,” are used interchangeably. Nevertheless, a material may be referred to as a cold storage material if the material has a melting point at or below 20° C., which is a standard condition, and may be referred to as a thermal storage material if the material has a melting point at or above 20° C.

(3) Thermal storage materials and cold storage materials are practical implementations of an aspect of the present invention and each contain either a combination of a thermal-storage base compound (cold-storage base compound) and a nucleating agent or a combination pf a thermal-storage base compound (cold-storage base compound), a nucleating agent, and an alkaline agent in an aspect of the present invention.

(4) The term, “thermal-storage base compound (cold-storage base compound),” refers to a composition, of water and a guest compound, that forms a semi-clathrate hydrate (as defined in (1) above) encaging a non-gaseous species as a guest. The thermal-storage base compound (cold-storage base compound) may be in the solid phase, in the liquid phase, or in a phase-changing state.

(5) The terms, “solidification temperature” and “freezing temperature,” both refer to a temperature at which a cold storage material changes from the liquid phase to the solid phase. In an aspect of the present invention, the solidification temperature, or the freezing temperature, is measured using a thermocouple while lowering the temperature of a cooling container (e.g., refrigerator, freezer, or programmable thermostatic chamber) housing a polypropylene bottle containing at least 50 mL of a cold storage material. It is known that supercooling phenomena can vary depending on the volume of the cold storage material. The inventors have confirmed through experiments that supercooling phenomena are hardly affected by the volume if the volume is greater than or equal to 50 mL.

(6) The onset temperature of melting is determined by extrapolating the temperature at which an exothermic peak starts toward a baseline on a differential scanning calorimetry (“DSC”) thermogram obtained by DSC.

(7) The terms, “frozen state” and “solidified state,” both refer to a state where the solid phase accounts for 95% or more of the total volume with the liquid phase, which is present in a tiny volume, being separated from the solid phase. The terms do not encompass a state where solid particles are suspended or dispersed in a liquid.

(8) Latent heat is calculated from the area of an exothermic peak on a DSC thermogram obtained by differential scanning calorimetry (DSC) and expressed as an amount of heat per weight or volume of the cold storage material.

(9) The terms, “positive hydration,” “hydrophobic hydration,” and “structure-forming hydration,” all refer to a state where water molecules around a cation are strongly attracted to the ion, thereby forming a highly ordered structure and being less likely to move than bulk water molecules. Clathrate hydration is hydrophobic hydration in the broad sense of the term.

(10) The terms, “negative hydration,” “hydrophilic hydration,” and “structure-breaking hydration,” all refer to a state where water molecules around a cation are attracted to the cation not as strongly as in positive hydration, but strongly enough to be separated from the hydrogen-bond network of bulk water molecules, thereby more likely to move than bulk water molecules.

(11) In thermal storage layers (cold storage layers) and transport media, solid particles of a clathrate encaging tetra-n-butylammonium bromide as a guest are generally often used in a dispersed or suspended state, or in the form of “slurry.” Most thermal storage materials (cold storage materials) used in the present embodiment change to solid, and do not turn into a suspended state, at or below the phase transition temperature. The heat that is available from one gram of an aqueous solution in a slurry state is as little as 7 to 11 calories, which is too little for the aqueous solution to be used as a thermal storage material (cold storage material). The thermal storage material (cold storage material) does not need to be in a suspended state at or below the phase transition temperature unless the usage of the material requires a fluid material. The thermal storage material (cold storage material) turns into a slurry when tetra-n-butylammonium bromide has a sufficiently low concentration, for example, 20 wt % or lower.

The following will describe embodiments of the present invention with reference to drawings.

### First Embodiment

### Composition of Cold Storage Materials

A cold storage material in accordance with the present invention changes phase at a prescribed temperature and contains water, a base compound, and a supercooling inhibitor. The base compound contains a quaternary ammonium salt and forms a semi-clathrate hydrate. Use of a base compound that forms a semi-clathrate hydrate renders large latent heat energy available for exploitation. The base compound is preferably tetrabutylammonium bromide (which may hereinafter be referred to as “TBAB”).

The supercooling inhibitor may be composed of (α) a pH adjuster forming cations that exhibit positive hydration and a nucleating agent that maintains alkalinity or (β) only a nucleating agent that maintains alkalinity.

### (α) Supercooling Inhibitor Containing Both pH Adjuster and Nucleating Agent

The pH adjuster is, for example, sodium carbonate, in which case the pH adjuster is aqueous and maintains alkalinity. The cold storage material preferably has a pH of 10 or higher, which makes it possible to prepare a sufficiently alkaline aqueous solution and form cations that exhibit positive hydration. The weight ratio of the pH adjuster to an aqueous solution composed of water and a base compound is preferably 2.0% (in the present embodiment, the aqueous solution is composed of water and TBAB). Sodium carbonate is easier to handle than sodium hydroxide because sodium carbonate is neither deleterious nor hazardous.

The nucleating agent is, for example, disodium hydrogen phosphate such as disodium hydrogen phosphate dihydrate, disodium hydrogen phosphate heptahydrate, or disodium hydrogen phosphate dodecahydrate and forms cations that exhibit positive hydration in an aqueous solution. Accordingly, these cations, formed in an aqueous solution maintained in an alkaline condition, exhibit positive hydration and serve as nuclei in solidification. That in turn raises solidification temperature, reducing difference between solidification temperature and melting temperature. The nucleating agent of this nature not only produces a tetragonal semi-clathrate hydrate, but unfailingly produces an orthorhombic semi-clathrate hydrate. The resultant cold storage material solidifies at or above 0° C.

The nucleating agent is preferably an anhydride or hydrate of disodium hydrogen phosphate and more preferably disodium hydrogen phosphate dodecahydrate. Containing both sodium carbonate and an anhydride or hydrate of disodium hydrogen phosphate in the aqueous solution allows stable solidification of the cold storage material. The weight ratio of the nucleating agent to an aqueous solution composed of water and a base compound is preferably 2.5% (in the present embodiment, the aqueous solution is composed of water and TBAB). This particular composition effectively suppresses supercooling.

### (β) Supercooling Inhibitor Containing Only Nucleating Agent

The supercooling inhibitor is, for example, sodium tetraborate, in which case the weight ratio of the sodium tetraborate to an aqueous solution composed of water and a base compound is preferably 2.0% (in the present embodiment, the aqueous solution is composed of water and TBAB).

### Method of Preparing Cold Storage Material

The cold storage material may be prepared by mixing water, a base compound, and a supercooling inhibitor at room temperature. A suitable amount of each component is weighed out before being mixed.

### Clathrate Hydrate

Clathrate hydrates typically have a polyhedral crystal structure (cage or basket) formed by hydrogen-bonded water molecules such as a dodecahedral, tetrakaidecahedral, or hexakaidecahedral structure. Water molecules are hydrogen bonded to each other to form a cavity and also to those water molecules forming another cavity, thereby forming a polyhedron. It is known that clathrate hydrates have crystal types called structure I and structure II.

Structure I has unit cells each formed of 46 water molecules, six large cavities (tetrakaidecahedra each of 12 five-membered rings and two six-membered rings), and two small cavities (tetrakaidecahedra each of five-membered rings). Meanwhile, structure II has unit cells each formed of 136 water molecules, eight large cavities (hexakaidecahedra each of 12 five-membered rings and four six-membered rings), and 16 small cavities (tetrakaidecahedra each of five-membered rings). These unit cells generally form a cubic crystal structure in clathrate hydrates encaging a gaseous species as a guest compound.

Meanwhile, when the guest compound is a large molecule of a non-gaseous species such as a quaternary ammonium salt used in the present invention, some hydrogen bonds forming a cage in the clathrate hydrate are broken, forming dangling bonds. Semi-clathrate hydrates encaging tetra-n-butylammonium bromide as a guest compound have two types of crystal structures: tetragonal (first hydrate) and orthorhombic (second hydrate).

An orthorhombic unit cell has six dodecahedral cages, four tetrakaidecahedral cages, and four pentakaidecahedral cages and encages two tetra-n-butylammonium bromide molecules as guest compounds. Bromine atoms are integrated into the cage structure and bonded to water molecules. Tetra-n-butylammonium ions (cations) are enclathrated in the center of four cages (two tetrakaidecahedral and two pentakaidecahedral cages) having some dangling bonds. The six dodecahedral cages are hollow. A tetragonal unit cell is similarly structured of a combination of dodecahedra, tetrakaidecahedra, and pentakaidecahedra, with the dodecahedra being hollow.

These two types of crystal structures are now described using hydration numbers (molar ratios) of tetra-n-butylammonium bromide and water. Water molecules have an average hydration number of approximately 26 (molar ratio of 1:26) in the tetragonal type and approximately 36 (molar ratio of 1:36) in the orthorhombic type. The concentration of tetra-n-butylammonium bromide in this condition is termed a congruent melting point composition, which is approximately 40 wt % in the tetragonal type and approximately 32 wt % in the orthorhombic type.

The drawings of the present application identify those samples containing disodium hydrogen phosphate and sodium carbonate as PC systems.

### Example 1

Tetrabutylammonium bromide (TBAB) was used as the base compound of a cold storage material. A 32-wt % aqueous TBAB solution was prepared from this TBAB. Disodium hydrogen phosphate dodecahydrate (nucleating agent) and sodium carbonate (pH adjuster) were added to the prepared 32-wt % aqueous TBAB solution in a weight ratio of 2.5% and 2.0% respectively relative to the 32-wt % aqueous TBAB solution, to obtain a cold storage material. The resultant 32-wt % aqueous TBAB solution contained both the first hydrate and the second hydrate.

### Example 2

TBAB was used as the base compound of a cold storage material. A 30-wt % aqueous TBAB solution was prepared from this TBAB. Disodium hydrogen phosphate dodecahydrate and sodium carbonate were added to the prepared 30-wt % aqueous TBAB solution in a weight ratio of 2.5% and 2.0% respectively relative to the 30-wt % aqueous TBAB solution, to obtain a cold storage material. The resultant 30-wt % aqueous TBAB solution contained both the first hydrate and the second hydrate.

### Example 3

TBAB was used as the base compound of a cold storage material. A 35-wt % aqueous TBAB solution was prepared from this TBAB. Disodium hydrogen phosphate dodecahydrate and sodium carbonate were added to the prepared 35-wt % aqueous TBAB solution in a weight ratio of 2.5% and 2.0% respectively relative to the 35-wt % aqueous TBAB solution, to obtain a cold storage material. The resultant 35-wt % aqueous TBAB solution contained both the first hydrate and the second hydrate.

### Example 4

TBAB was used as the base compound of a cold storage material. A 38-wt % aqueous TBAB solution was prepared from this TBAB. Disodium hydrogen phosphate dodecahydrate and sodium carbonate were added to the prepared 38-wt % aqueous TBAB solution in a weight ratio of 2.5% and 2.0% respectively relative to the 38-wt % aqueous TBAB solution, to obtain a cold storage material. The resultant 38-wt % aqueous TBAB solution contained only the first hydrate.

### Example 5

TBAB was used as the base compound of a cold storage material. A 40-wt % aqueous TBAB solution was prepared from this TBAB. Disodium hydrogen phosphate dodecahydrate and sodium carbonate were added to the prepared 40-wt % aqueous TBAB solution in a weight ratio of 2.5% and 2.0% respectively relative to the 40-wt % aqueous TBAB solution, to obtain a cold storage material. The resultant 40-wt % aqueous TBAB solution contained only the first hydrate.

### Example 6

Tetrabutylammonium bromide (TBAB) was used as the base compound of a cold storage material. A 32-wt % aqueous TBAB solution was prepared from this TBAB. Sodium tetraborate pentahydrate (supercooling inhibitor) was added to the prepared 32-wt % aqueous TBAB solution in a weight ratio of 2.0% relative to the 32-wt % aqueous TBAB solution, to obtain a cold storage material. The resultant 32-wt % aqueous TBAB solution contained both the first hydrate and the second hydrate.

### Example 7

TBAB was used as the base compound of a cold storage material. A 30-wt % aqueous TBAB solution was prepared from this TBAB. Sodium tetraborate pentahydrate was added to the prepared 30-wt % aqueous TBAB solution in a weight ratio of 2.0% relative to the 30-wt % aqueous TBAB solution, to obtain a cold storage material. The resultant 30-wt % aqueous TBAB solution contained both the first hydrate and the second hydrate.

### Example 8

TBAB was used as the base compound of a cold storage material. A 35-wt % aqueous TBAB solution was prepared from this TBAB. Sodium tetraborate pentahydrate was added to the prepared 35-wt % aqueous TBAB solution in a weight ratio of 2.0% relative to the 35-wt % aqueous TBAB solution, to obtain a cold storage material. The resultant 35-wt % aqueous TBAB solution contained both the first hydrate and the second hydrate.

### Example 9

TBAB was used as the base compound of a cold storage material. A 38-wt % aqueous TBAB solution was prepared from this TBAB. Sodium tetraborate pentahydrate was added to the prepared 38-wt % aqueous TBAB solution in a weight ratio of 2.0% relative to the 38-wt % aqueous TBAB solution, to obtain a cold storage material. The resultant 38-wt % aqueous TBAB solution contained both the first hydrate and the second hydrate.

### Example 10

TBAB was used as the base compound of a cold storage material. A 40-wt % aqueous TBAB solution was prepared from this TBAB. Sodium tetraborate pentahydrate was added to the prepared 40-wt % aqueous TBAB solution in a weight ratio of 2.0% relative to the 40-wt % aqueous TBAB solution, to obtain a cold storage material. The resultant 40-wt % aqueous TBAB solution contained only the first hydrate.

### 1. Comparison of Freezing Temperatures

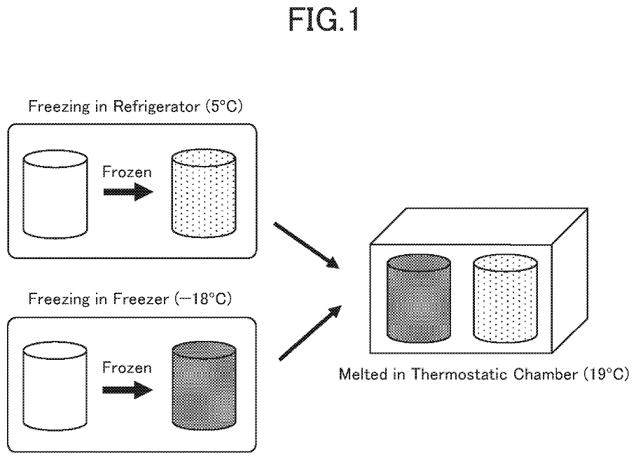

FIG. 1 is a schematic illustration of procedures of comparing freezing temperatures. Referring to FIG. 1, the cold storage materials obtained in Examples 1 and 6 were put into respective containers and frozen at two different temperatures in a refrigerator (5° C.) and in a freezer (−18° C.). After the freezing, the containers were placed in a thermostatic chamber maintained at a constant temperature of 19° C., to measure changes in temperature of the cold storage materials.

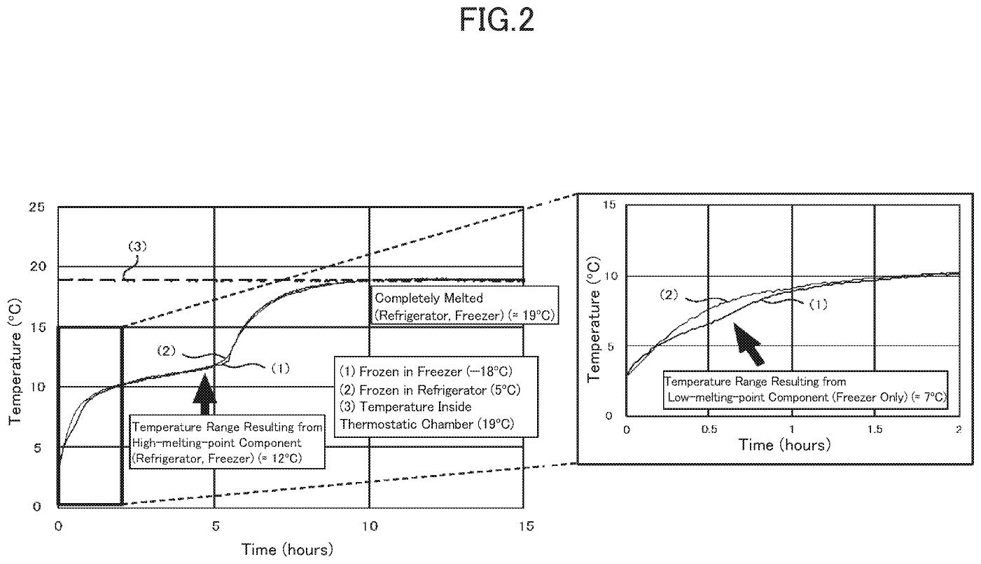

FIG. 2 is a graph representing changes in temperature of samples of the cold storage material obtained in Example 1 that were frozen at two different temperatures and then placed in a thermostatic chamber. FIG. 2 shows that the cold storage material sample frozen in a freezer (−18° C.) has, at temperatures from 5° C. to 10° C., a different temperature range (approximately 7° C.) resulting from a low-melting-point component than does the cold storage material sample frozen in a refrigerator (5° C.).

Next, the cold storage material obtained in Example 1 was subjected to differential scanning calorimetry (DSC). The temperature setting was (a) lowered from 30° C. to −30° C. (5° C./min), held at −30° C. for 5 minutes, and then raised to 30° C. (5° C./min) (equivalent to freezing in a freezer) and (b) lowered from 30° C. to 3° C. (5° C./min), held at 3° C. for 100 minutes, and then raised to 30° C. (5° C./min) (equivalent to freezing in a refrigerator). Latent heat was calculated from an area drawn from melting data.

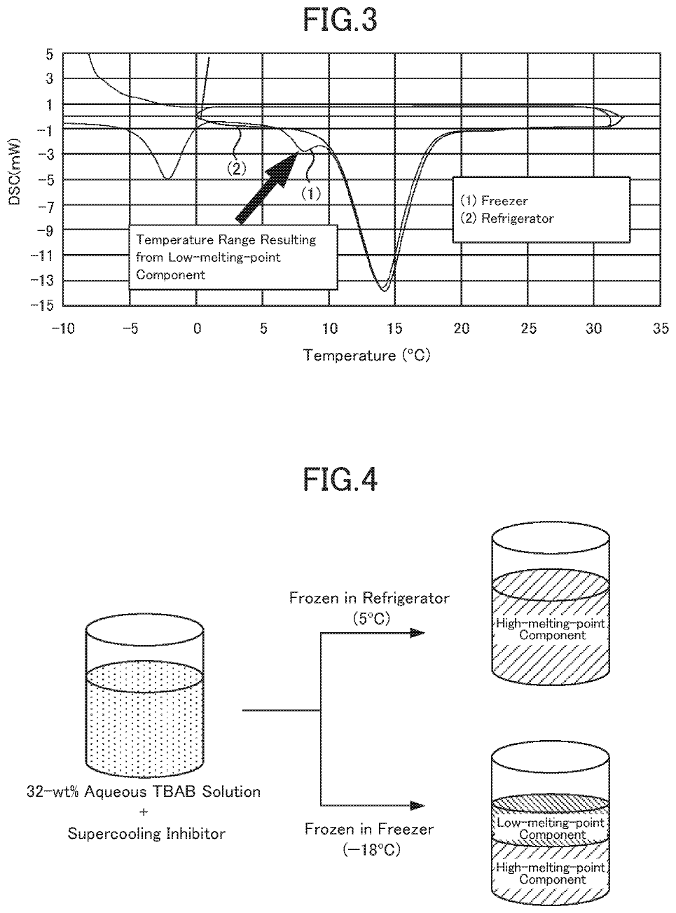

FIG. 3 is a graph representing results of a DSC experiment on the cold storage material obtained in Example 1. FIG. 3 shows that the cold storage material frozen at a temperature equivalent to freezing in a freezer has a temperature range resulting from a low-melting-point component.

FIG. 4 is a schematic illustration of the arrangement of a high-melting-point component and a low-melting-point component that result from different freezing temperatures. FIG. 4 shows that the 32-wt % aqueous TBAB solution, if frozen in a refrigerator (5° C.), produces only a high-melting-point component and if frozen in a freezer (−18° C.), produces both a high- and low-melting-point components. FIG. 4 depicts the two high- and low-melting-point components separately for ease in understanding. In practice, however, a mixture of the high- and low-melting-point components form from the aqueous TBAB solution.

Samples of the cold storage material obtained in Example 1 were put into containers, frozen respectively at 0° C., −5° C., −10° C., and −15° C., and then placed in a thermostatic chamber maintained at a constant temperature of 19° C., to measure changes in temperature of the cold storage material samples.

FIG. 5 is a graph representing changes in temperature of the cold storage material samples frozen at 0° C., −5° C., −10° C., −15° C., and −18° C. FIG. 5 shows that the cold storage material samples frozen at or below −10° C. have a temperature range resulting from a low-melting-point component.

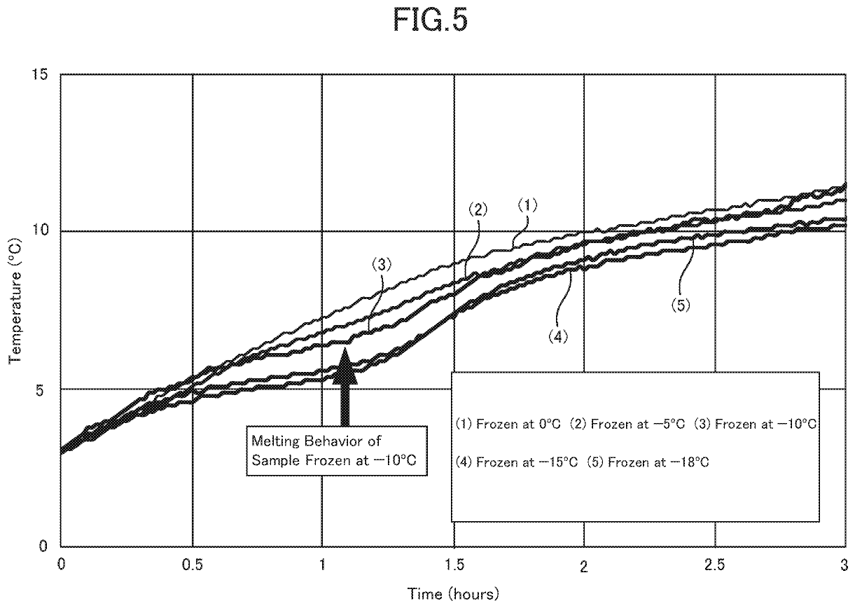

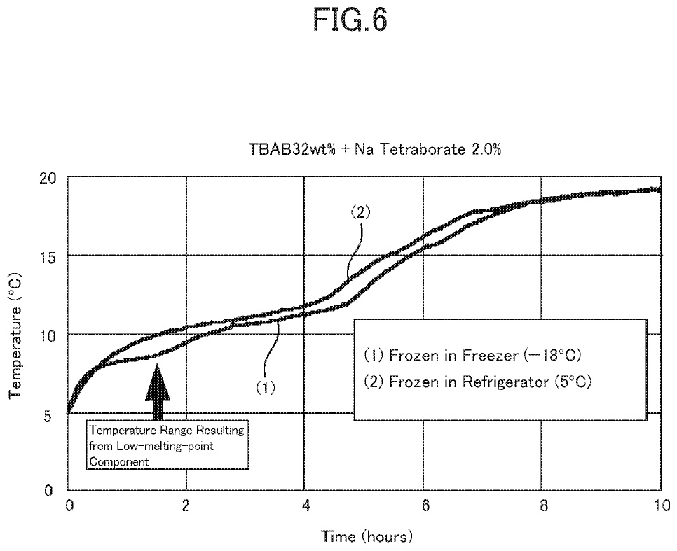

FIG. 6 is a graph representing changes in temperature of samples of the cold storage material obtained in Example 6 that were frozen at two different temperatures and then placed in a thermostatic chamber. FIG. 6 shows that the cold storage material obtained in Example 6 exhibits a similar behavior, or more specifically, the cold storage material sample frozen in a freezer (−18° C.) has a different temperature range resulting from a low-melting-point component than does the cold storage material sample frozen in a refrigerator (5° C.).

### 2. Comparison of Concentrations

Samples were prepared with various concentrations of the base compound (TBAB) and subjected to differential scanning calorimetry (DSC). The melting temperature range of each cold storage material was checked in an endothermic reaction that occurs in melting in DSC experimentation. The temperature setting was lowered from 30° C. to −30° C. (5° C./min), held at −30° C. for 5 minutes, and then raised to 30° C. (5° C./min) (equivalent to freezing in a freezer). Latent heat was calculated from an area drawn from melting data.

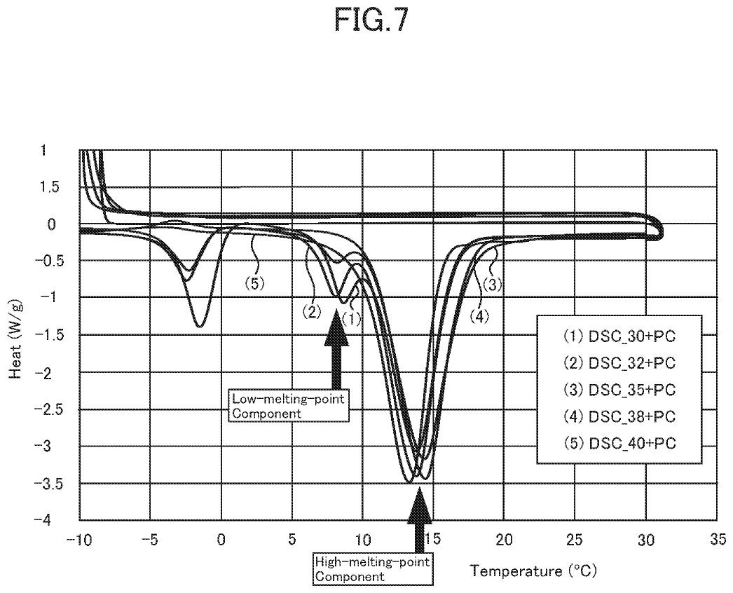

FIG. 7 is a graph representing results of a DSC experiment on the cold storage materials obtained in Examples 1 to 5. In the legend for curves (1) to (5), 30, 32, 35, 38, and 40 denote TBAB concentrations in mass %, P denotes disodium hydrogen phosphate dodecahydrate, and C denotes sodium carbonate. Hence, for example, “DSC_30+PC” denotes a 30-wt % (-mass %) TBAB solution additionally containing 2.5% disodium hydrogen phosphate and 2.0% sodium carbonate. FIG. 7 shows that the cold storage materials (30-wt %, 32-wt %, and 35-wt % TBAB solutions) obtained in Examples 1 to 3 each contain a low-melting-point component and a high-melting-point component.

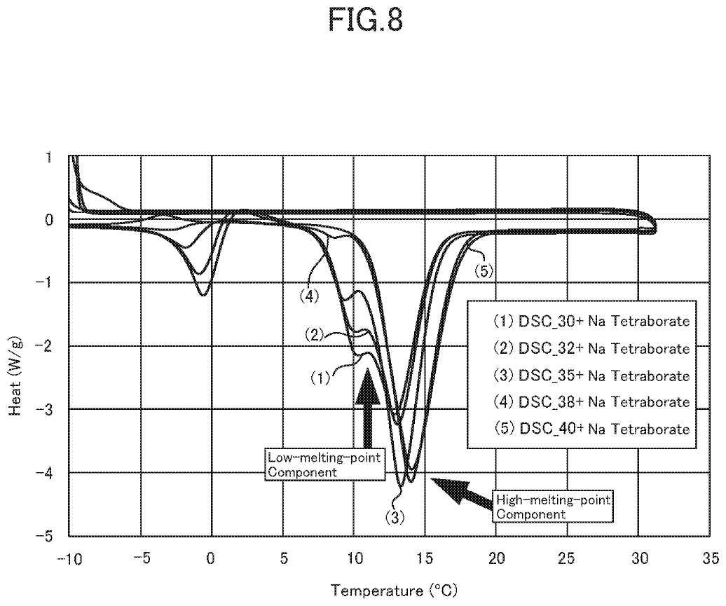

FIG. 8 is a graph representing results of a DSC experiment on the cold storage materials obtained in Examples 6 to 10. In the legend for curves (1) to (5), 30, 32, 35, 38, and 40 denote TBAB concentrations in mass %, and Na tetraborate denotes sodium tetraborate pentahydrate. Hence, for example, “DSC_30+Na tetraborate” denotes a 30-wt % (-mass %) TBAB solution additionally containing 2.0% sodium tetraborate pentahydrate. FIG. 8 shows that the cold storage materials (30-wt %, 32-wt %, 35-wt %, and 38-wt % TBAB solutions) obtained in Examples 6 to 9 each contain a low-melting-point component and a high-melting-point component. FIG. 17 shows low-melting-point components and high-melting-point components produced by TBAB 32 wt %+Na tetraborate for different freezing temperatures.

The first embodiment, as described so far, provides cold storage materials that have a plurality of different melting points.

### Second Embodiment

### Structure of Cold Storage Packs

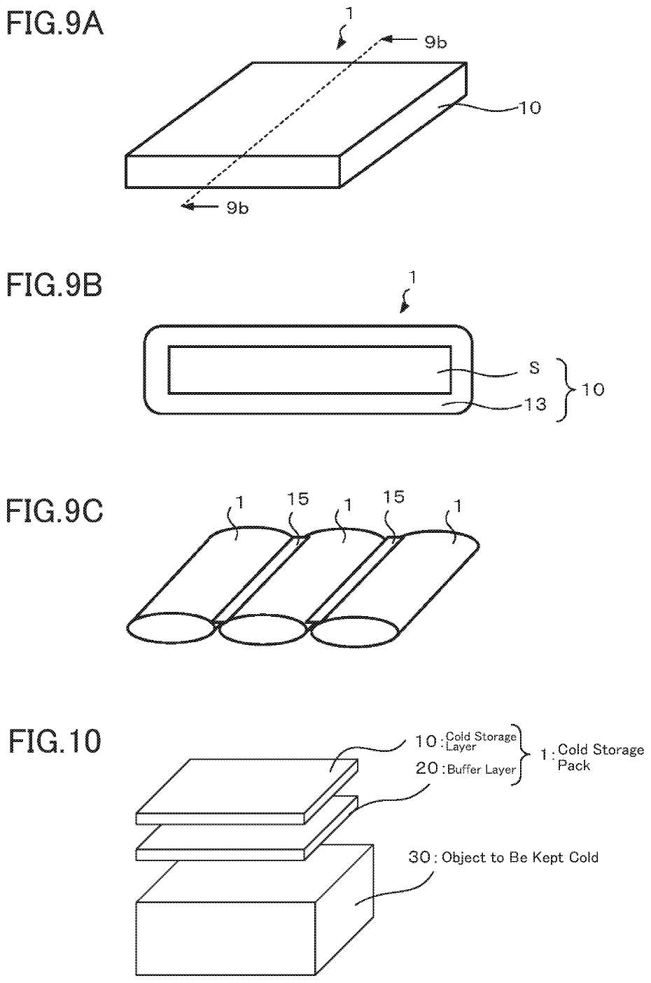

FIG. 9A is a schematic perspective view of a cold storage pack in accordance with the present embodiment. FIG. 9B is a cross-sectional view taken along line 9b-9b in FIG. 9A. A cold storage pack 1 (hereinafter, may be referred to as a cold insulation pack) includes a cold storage layer 10 of aforementioned cold storage material S, the storage layer 10 being packaged in a film 13. There may be provided a plurality of cold storage packs 1 coupled together by articulation mechanisms 15 as shown in FIG. 9C.

FIG. 10 is a schematic perspective view of Variation Example 1 of the cold storage pack in accordance with the present embodiment. Referring to FIG. 10, the cold storage pack (cold insulation pack) 1 may further include a buffer layer 20 containing a material that is not frozen at the temperature at which the cold storage layer 10 is frozen (the material may hereinafter be referred to as an antifreeze material), so that the cold storage pack 1 includes the cold storage layer 10 of cold storage material S and the buffer layer 20 containing an antifreeze material. The buffer layer 20 is brought into contact with an object-to-be-kept-cold 30, in order to transfer heat between the object-to-be-kept-cold 30 and the cold storage layer 10. This arrangement allows for alleviation of heat deprivation (reduction of heat removed from the object to be kept cold). Still referring to FIG. 10, since the antifreeze material in the buffer layer 20 is in liquid phase at the phase transition temperature of the cold storage material in the cold storage layer 10, and the buffer-layer-packaging member is flexible, the buffer layer 20 attaches well to the object-to-be-kept-cold 30. The buffer layer 20 may be replaced by a plurality of cold storage layers 10 and buffer layers 20 coupled together by articulation mechanisms 15. Attachment of the buffer layer 20 to the object-to-be-kept-cold 30 may be improved by adding a thickening agent to the buffer layer 20 for thickening and making it easier to preserve shape.

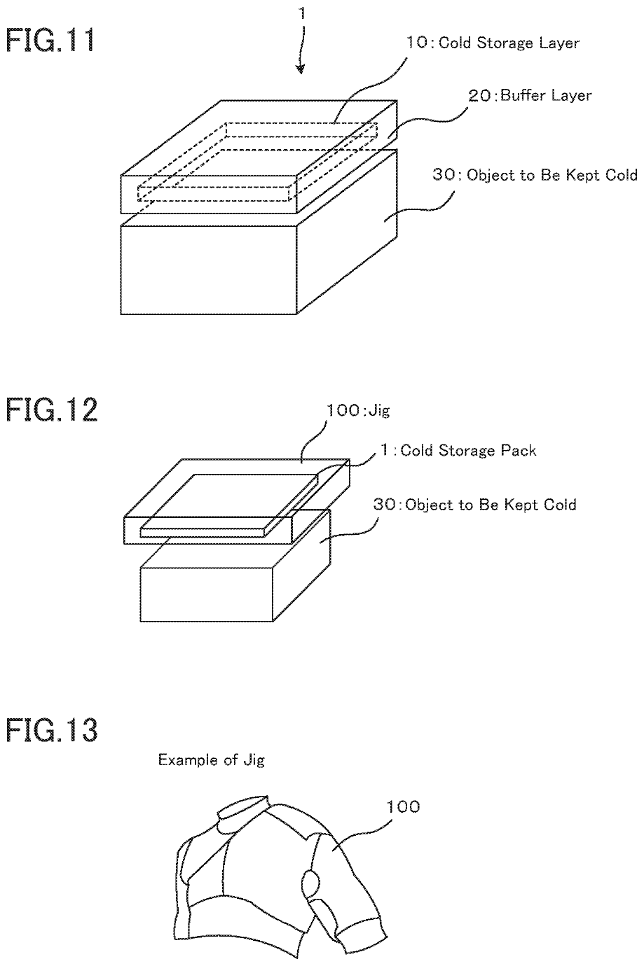

FIG. 11 is a schematic perspective view of Variation Example 2 of the cold storage pack in accordance with the present embodiment. Referring to FIG. 11, the cold storage pack (cold insulation pack) 1 may be structured such that the buffer layer 20 encases the entire cold storage layer 10 like a pack-in-pack.

The cold storage pack (cold insulation pack) 1, when used, may be enclosed in a jig 100 for fixing the cold storage pack 1 to the object-to-be-kept-cold 30 as shown in FIG. 12. Alternatively, the cold storage pack (cold insulation pack) 1, when used, may be fixed using the jig 100. The jig 100 may be, for example, a supporter or a towel (see FIG. 13).

The second embodiment, as described so far, enables a single cold insulation pack (cooling medium) to maintain an object at suitable, low temperature in accordance with a situation through adjustment of freezing temperature. In particular, if the cold insulation pack is frozen at a temperature above −10° C., the cold storage material comes to have a relatively high melting point. The cold insulation pack in accordance with the present embodiment, if used for precooling prior to physical exercise or activity in a hot environment, allows for gentle cooling of the human body.

### Third Embodiment

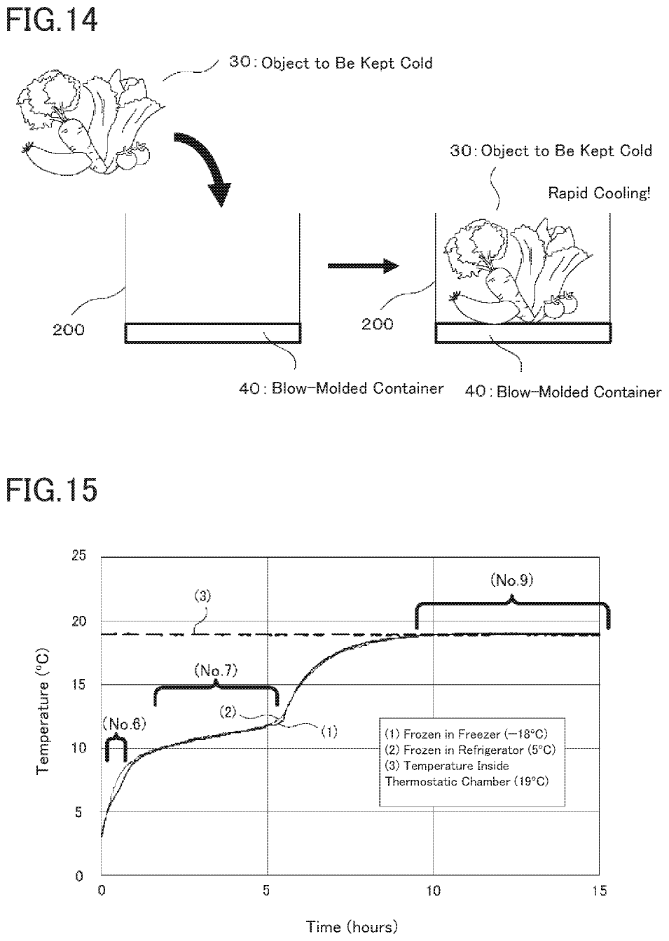

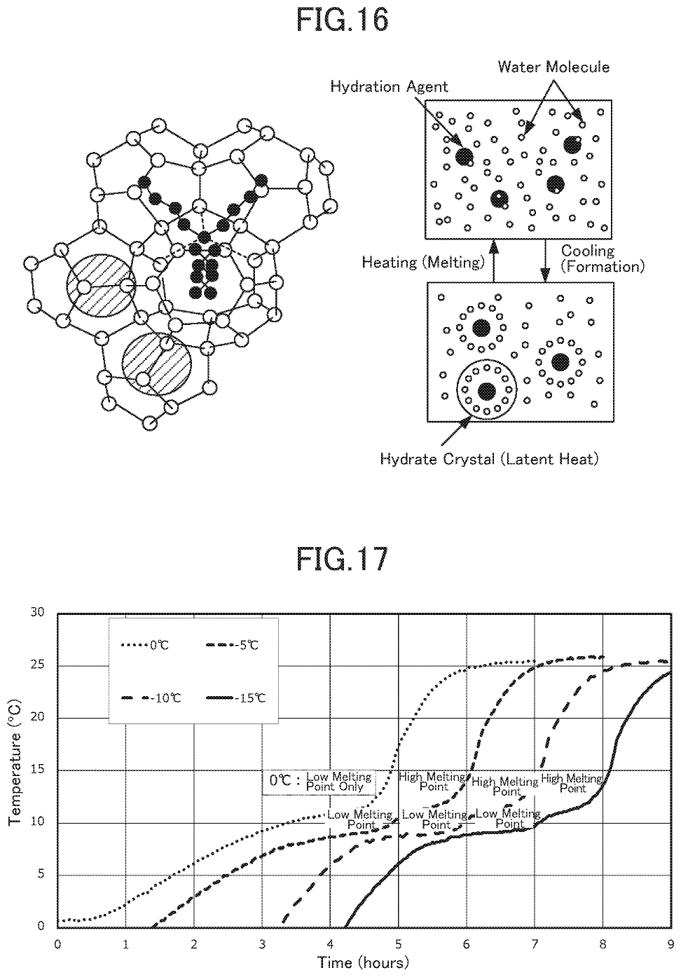

The cold storage materials described above may be used in a transport container 200. For example, a cold storage material may be put into a blow-molded container 40 to construct a cold insulator as shown in FIG. 14. A cold insulator, including the blow-molded container 40 and a cold storage material in the blow-molded container 40, may be frozen at or below −10° C. and placed in inside the transport container 200 for transporting, for example, food. Using the cold insulation pack when a large quantity of heat flows into the food in a hot environment can achieve rapid removal of heat and enables the inside of the transport container 200 to be subsequently maintained at a constant temperature for a period of time.

The third embodiment, as described so far, enables a single cold insulation pack (cooling medium) to maintain an object at suitable, low temperature in accordance with a situation through adjustment of freezing temperature. In particular, if the cold insulation pack is frozen at or below −10° C., the cold storage material comes to have a relatively low melting point, which in turn enables rapid cooling of an object.

### Thermochromic Substance

The cold storage material described above and the cold storage pack and transport container containing the cold storage material may advantageously contain a thermochromic substance. Thermochromic substances change color with temperature, have various temperature ranges, colors, and forms, and are commercially available now as listed in Table 1.

TABLE 1

| | |
|---|---|
| Capsule Slurry | May be mixed with a fixing agent in |
| | dispersion water to prepare a water-based |
| | paint or ink. This paint or ink may be |
| | absorbed, for example, by blank T-shirts so |
| | that the clothes can change color with body |
| | temperature. |
| Capsule Powder | Fine (several micrometers) particulate powder. |
| | May be mixed with an oil-based binder (fixing |
| | agent) to prepare an ink for printing on, for |
| | example, film, glass, and metal. |
| Master Batch | Commercially sold as pellets of polypropylene |
| | or a like plastic. The resin may be increased in |
| | volume by 5 to 10 folds and injection-molded |
| | to make, for example, bath toys, |
| | color-pattern-changing mugs, |
| | temperature-indicating containers for frozen |
| | food, and cold drink containers indicating |
| | whether the drink is ready to drink. Injection |
| | mold temperature is 200° C. or lower. |
| Aqueous Screen Ink | Suitable for printing on T-shirts or like clothes. |
| | Best if an 80- to 100-mesh screen is used. Best |
| | if subjected to heat drying at 110° C. to 120° C. |
| | for approximately 3 minutes or at 150° C. to |
| | 160° C. for approximately 1 minute after |
| | printing. |
| Oil-based Screen Ink | Suitable for printing on, for example, plastic |
| | films. Best if an 80- to 100-mesh screen is |
| | used. Best if subjected to heat drying at 40° C. |
| | to 60° C. for approximately 1 hour after |
| | printing. |
| Aqueous Ink | Suitable for printing on, for example, paper |
| | and cloth. Best if subjected to heat drying at |
| | 110° C. to 120° C. for approximately 3 minutes |
| | or at 150° C. to 160° C. for approximately 1 |
| | minute after printing. |
| Oil-based Ink | Suitable for printing on, for example, plastic |
| | films. Best if subjected to heat drying at 40° C. |
| | to 60° C. for approximately 3 minutes after |
| | printing. |

Table 2 is a list of exemplary color-changing temperatures of commercially available thermochromic substances. For instance, if thermochromic substances Nos. 6, 7, and 9 in Table 2 are used in the cold storage material of Example 1, thermochromic substance No. 6 changes color in a temperature range resulting from a low-melting-point component, thermochromic substance No. 7 changes color in a temperature range resulting from a high-melting-point component, and thermochromic substance No. 10 changes color in a temperature range where cooling effect is no longer available. The user can visually recognize the current temperature range from these color changes. These color changes of thermochromic substances with the temperature of the cold storage material enable the user to visually recognize the temperature condition of the cold storage material.

TABLE 2

| | Color-changing Temperature | | | | | | | |
|---|---|---|---|---|---|---|---|---|
| No. | Colored | | | Intermediate Color | | | Discolored | |
| 1 | −25° | C. | ←→ | −20° | C. | ←→ | −15° | C. |
| 2 | −20° | C. | ←→ | −15° | C. | ←→ | −10° | C. |
| 3 | −15° | C. | ←→ | −10° | C. | ←→ | −5° | C. |
| 4 | −10° | C. | ←→ | −5° | C. | ←→ | 0° | C. |
| 5 | −5° | C. | ←→ | 0° | C. | ←→ | 5° | C. |
| 6 | 0° | C. | ←→ | 5° | C. | ←→ | 10° | C. |
| 7 | 5° | C. | ←→ | 10° | C. | ←→ | 15° | C. |
| 8 | 10° | C. | ←→ | 15° | C. | ←→ | 20° | C. |
| 9 | 15° | C. | ←→ | 20° | C. | ←→ | 25° | C. |

(A) The present invention, in one aspect thereof, may be arranged as follows. Specifically, the present invention, in one aspect thereof, is directed to a cold storage material changing phase at a prescribed temperature, the cold storage material including: water; a base compound including a quaternary ammonium salt that forms a semi-clathrate hydrate; and a supercooling inhibitor that suppresses supercooling.

According to this arrangement, the cold storage material has one or more melting points depending on a temperature range in which the cold storage material has been frozen, which allows the user to change the freezing temperature in view of intended usage so that the cold storage material has a desirable melting point to the user. In addition, the use of a base compound that forms a semi-clathrate hydrate renders large latent heat energy available for exploitation.

(B) In the cold storage material in accordance with an aspect of the present invention, the supercooling inhibitor includes: a nucleating agent forming cations that exhibit positive hydration; and a pH adjuster that maintains alkalinity.

According to this arrangement, the aqueous solution remains alkaline, enabling production of cations that exhibit positive hydration. The cations raise solidification temperature, thereby reducing difference between solidification temperature and melting temperature. As a result, supercooling is further suppressed.

(C) In the cold storage material in accordance with an aspect of the present invention, the base compound is tetrabutylammonium bromide, the nucleating agent is an anhydride or hydrate of disodium hydrogen phosphate, the pH adjuster is sodium carbonate, and the cold storage material has a first melting point and a second melting point if the cold storage material has been frozen at or below −10° C., the first melting point differing from the second melting point.

According to this arrangement, the cold storage material forms a low-melting-point component that has a first melting point and a high-melting-point component that has a second melting point higher than the low-melting-point component when the cold storage material is frozen at or below −10° C. The low-melting-point component that has the first melting point provides rapid cooling effects. In addition, the cold storage material solidifies in a stable manner because the cold storage material contains both sodium carbonate and an anhydride or hydrate of disodium hydrogen phosphate. Supercooling suppressing effects can be improved without having to decrease the latent heat energy of the base compound.

(D) In the cold storage material in accordance with an aspect of the present invention, the water and the tetrabutylammonium bromide form an aqueous solution with a concentration of from 30 wt % to 35 wt %, both inclusive, the anhydride or hydrate of disodium hydrogen phosphate accounts for 2.5% in weight of the aqueous solution, and the sodium carbonate accounts for 2.0% in weight of the aqueous solution.

By thus setting the concentration of an aqueous solution containing water and tetrabutylammonium bromide and the weight ratios of materials to this aqueous solution, a cold storage material can be produced that has one or two melting points depending on freezing temperature.

(E) In the cold storage material in accordance with an aspect of the present invention, the base compound is tetrabutylammonium bromide, the supercooling inhibitor is an anhydride or hydrate of sodium tetraborate, and the cold storage material has a first melting point and a second melting point if the cold storage material has been frozen at or below −5° C., the first melting point differing from the second melting point.

According to this arrangement, the cold storage material forms a low-melting-point component that has a first melting point and a high-melting-point component that has a second melting point higher than the low-melting-point component when the cold storage material is frozen at or below −5° C. The low-melting-point component that has the first melting point provides rapid cooling effects. In addition, the cold storage material solidifies in a stable manner because the cold storage material contains sodium tetraborate. Supercooling suppressing effects can be improved without having to decrease the latent heat energy of the base compound.

(F) In the cold storage material in accordance with an aspect of the present invention, the water and the tetrabutylammonium bromide form an aqueous solution with a concentration of from 30 wt % to 38 wt %, both inclusive, and the sodium tetraborate accounts for 2.0% in weight of the aqueous solution.

By thus setting the concentration of an aqueous solution containing water and tetrabutylammonium bromide and the weight ratios of materials to this aqueous solution, a cold storage material can be produced that has one or two melting points depending on freezing temperature.

(G) In the cold storage material in accordance with an aspect of the present invention, the cold storage material has only the second melting point if the cold storage material has been frozen at a temperature above −5° C. or −10° C.

The cold storage material forms only a high-melting-point component that has the second melting point when the cold storage material is frozen at a temperature higher than −5° C. or −10° C. The high-melting-point component that has the second melting point provides gentle cooling effects.

(H) The present invention, in one aspect thereof, is directed to a cold storage pack including: the cold storage material of any one of (A) to (G); and a packaging member covering the cold storage material.

The single cold storage material, having two melting points, can be used with a melting temperature that is suitable for intended usage. More specifically, if the user wants to cool an object rapidly, the user can do so by using the low-melting-point component, which is formed when the cold storage material is frozen at or below −5° C. or −10° C.; if the user wants to cool an object gradually, the user can do so by using the high-melting-point component, which is formed when the cold storage material is frozen at 5° C.

### INDUSTRIAL APPLICABILITY

Some aspects of the present invention are applicable, for example, to cold storage materials and cold storage packs, where a single cooling medium (cold insulation pack) containing a cold storage material having a plurality of melting points can keep an object at a low temperature that is suited to a situation.

1. A cold storage material changing phase at a prescribed temperature, the cold storage material comprising:
water;
a base compound comprising a quaternary ammonium salt that forms a semi-clathrate hydrate; and
a supercooling inhibitor that suppresses supercooling, wherein
the cold storage material has one or more melting points depending on a temperature range in which the cold storage material has been frozen.

2. The cold storage material according to claim 1, wherein the supercooling inhibitor comprises: a nucleating agent forming cations that exhibit positive hydration; and a pH adjuster that maintains alkalinity.

3. The cold storage material according to claim 2, wherein
the base compound is tetrabutylammonium bromide,
the nucleating agent is an anhydride or hydrate of disodium hydrogen phosphate,
the pH adjuster is sodium carbonate, and
the cold storage material has a first melting point and a second melting point if the cold storage material has been frozen at or below −10° C., the first melting point differing from the second melting point.

4. The cold storage material according to claim 3, wherein
the water and the tetrabutylammonium bromide form an aqueous solution with a concentration of from 30 wt % to 35 wt %, both inclusive,
the anhydride or hydrate of disodium hydrogen phosphate accounts for 2.5% in weight of the aqueous solution, and
the sodium carbonate accounts for 2.0% in weight of the aqueous solution.

5. The cold storage material according to claim 4, wherein the nucleating agent is disodium hydrogen phosphate dodecahydrate.

6. The cold storage material according to claim 1, wherein
the base compound is tetrabutylammonium bromide,
the supercooling inhibitor is an anhydride or hydrate of sodium tetraborate, and
the cold storage material has a first melting point and a second melting point if the cold storage material has been frozen at or below −5° C., the first melting point differing from the second melting point.

7. The cold storage material according to claim 6, wherein
the water and the tetrabutylammonium bromide form an aqueous solution with a concentration of from 30 wt % to 38 wt %, both inclusive, and
the sodium tetraborate is pentahydrate and accounts for 2.0% in weight of the aqueous solution.

8. The cold storage material according to claim 3, wherein the cold storage material has only the second melting point if the cold storage material has been frozen at a temperature above −10° C.

9. The cold storage material according to claim 6, wherein the cold storage material has only the second melting point if the cold storage material has been frozen at a temperature above −5° C.

10. A cold storage pack comprising:
the cold storage material according to claim 1; and
a packaging member covering the cold storage material.
